(19) United States
(12) Patent Application Publication
(10) Pub. No.: US 2011/0022060 A1
(43) Pub. Date: Jan. 27, 2011

(54) SURGICAL ROBOT

(76) Inventors: Jong Seok Won, Gyeonggi-do (KR); Seung Wook Choi, Gyeonggi-do (KR); Kwang Ha, Gyeonggi-do (KR)

Correspondence Address: BIRCH STEWART KOLASCH & BIRCH, PO BOX 747, FALLS CHURCH, VA, 22040-0747, US

(21) Appl. No.: 12/673,725

(22) PCT Filed: Mar. 18, 2009
(86) PCT No.: PCT/KR09/01381

§ 371 Date: Feb. 16, 2010

(30) Foreign Application Priority Data
Jul. 25, 2008 (KR) 10-2008-0072707

(51) Int. Cl. A61B 19/00

(52) U.S. Cl. 606/130

(57) ABSTRACT

A surgical robot is disclosed, which includes: a handle unit; a main body connected to the handle unit; a robot arm connected to the main body and configured to operate in correspondence with a first manipulation on the handle unit; an instrument mounted on a front end of the robot arm; and a manipulation unit coupled to a far end of the instrument and configured to perform an action required for surgery in correspondence with a second manipulation on the handle unit, wherein the main body is coupled to a support unit. The surgical robot can be constructed with a simpler, slimmer structure, by having the arms of the surgical robot moved and rotated manually and having the instruments manipulated by the robot. Thus, the surgical robot can be installed in a narrow space, and the costs for manufacturing and installing the robot can be reduced. In particular, in the case of simple surgical procedures, the operator may readily manipulate the surgical robot next to the patient, for improved applicability and reliability.

### CROSS-REFERENCE TO RELATED APPLICATIONS

This application claims foreign priority benefits under 35 U.S.C. .sctn. 119(a)-(d) to PCT/KR2009/001381, filed Mar. 18, 2009, which is hereby incorporated by reference in its entirety.

### BACKGROUND

1. Technical Field

The present invention relates to a surgical robot.

2. Description of the Related Art

In the field of medicine, surgery refers to a procedure in which a medical device is used to make a cut or an incision in or otherwise manipulate a patient's skin, mucosa, or other tissue, to treat a pathological condition. A surgical procedure such as a laparotomy, etc., in which the skin is cut open and an internal organ, etc., is treated, reconstructed, or excised, may entail problems of blood loss, side effects, pain, and scars, and as such, the use of robots is currently regarded as a popular alternative.

A conventional surgical robot may include a master robot, which is manipulated by the doctor to generate and transmit the necessary signals, and a slave robot, which receives the signals from the master robot to actually apply the manipulation to the patient. Typically, the slave robot may be mounted in the operating room, and the master robot may be mounted in a manipulation room, with the master robot and slave robot connected by a wired and/or wireless system to allow remote operation of a surgical procedure.

However, a conventional surgical robot may have a very large volume, requiring a considerable amount of space for installation, and may include a complicated set of apparatus, requiring a lot of time and cost in manufacturing, installing, and training for the apparatus. In particular, using a conventional surgical robot for an otherwise simple surgical procedure can be inefficient, due to the time and cost involved in robot surgery.

The information in the background art described above was obtained by the inventors for the purpose of developing the present invention or was obtained during the process of developing the present invention. As such, it is to be appreciated that this information did not necessarily belong to the public domain before the patent filing date of the present invention.

### SUMMARY

An aspect of the present invention is to provide a surgical robot having a simpler and slimmer structure, so that the surgical robot may be installed in a narrow space, the costs for manufacturing and installing the robot may be reduced, and the operator may be enabled to easily manipulate the robot.

Other technical problems addressed by the present invention will be readily understood from the descriptions that follow.

One aspect of the present invention provides a surgical robot that includes: a handle unit; a main body connected to the handle unit; a robot arm connected to the main body and configured to operate in correspondence with a first manipulation on the handle unit; an instrument mounted on a front end of the robot arm; and a manipulation unit coupled to a far end of the instrument and configured to perform an action required for surgery in correspondence with a second manipulation on the handle unit, wherein the main body is coupled to a support unit.

The main body can be coupled to the support unit such that the main body is able to move and rotate, and can be configured to move and rotate in correspondence with a third manipulation on the handle unit. One end of the support unit can be installed on the floor or the ceiling of the operating room in which the surgical robot is installed, and can be formed as a movable self-standing frame in the operating room. On the portion of the main body that is coupled to the support unit, a power supply terminal can be formed, through which the main body may be supplied with electrical power. A laparoscope can be additionally mounted on the front end of the robot arm, and a monitor can be installed on the main body to output an image signal transmitted from the laparoscope.

The support unit can be formed as a hand guide that includes the handle unit and is configured to fit onto a user's hand, where the second manipulation can be performed in accordance to a movement of the finger and/or wrist of the user's hand on which the hand guide is fitted. In this case, an arm guide can be connected to the hand guide, and the first manipulation can be performed in accordance to a movement of the user's arm.

An arm guide, which includes the handle unit, and which is configured to fit onto a user's arm, can be coupled to the main body, and the first manipulation can be performed in accordance to a movement of the user's arm on which the hand guide is fitted.

Additional aspects, features, and advantages, other than those described above, will be obvious from the claims and written description below.

According to an embodiment of the present invention, a surgical robot can be constructed with a simpler, slimmer structure, by having the arms of the surgical robot moved and rotated manually and having the instruments manipulated by the robot. Thus, the surgical robot can be installed in a narrow space, and the costs for manufacturing and installing the robot can be reduced. In particular, in the case of simple surgical procedures, the operator may readily manipulate the surgical robot next to the patient, for improved applicability and reliability.

### BRIEF DESCRIPTION OF THE DRAWINGS

FIG. 1 is a perspective view schematically illustrating a surgical robot according to an embodiment of the present invention.

FIG. 2 is a perspective view schematically illustrating a surgical robot according to another embodiment of the present invention.

### DETAILED DESCRIPTION

As the present invention allows for various changes and numerous embodiments, particular embodiments will be illustrated in the drawings and described in detail in the written description. However, this is not intended to limit the present invention to particular modes of practice, and it is to be appreciated that all changes, equivalents, and substitutes that do not depart from the spirit and technical scope of the present invention are encompassed in the present invention. In the written description, certain detailed explanations of related art are omitted when it is deemed that they may unnecessarily obscure the essence of the present invention.

While such terms as “first” and “second,” etc., may be used to describe various components, such components must not be limited to the above terms. The above terms are used only to distinguish one component from another.

The terms used in the present specification are merely used to describe particular embodiments, and are not intended to limit the present invention. An expression used in the singular encompasses the expression of the plural, unless it has a clearly different meaning in the context. In the present specification, it is to be understood that the terms “including” or “having,” etc., are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added.

Certain embodiments of the present invention will be described below in detail with reference to the accompanying drawings. Those components that are the same or are in correspondence are rendered the same reference numeral regardless of the figure number, and redundant descriptions are omitted.

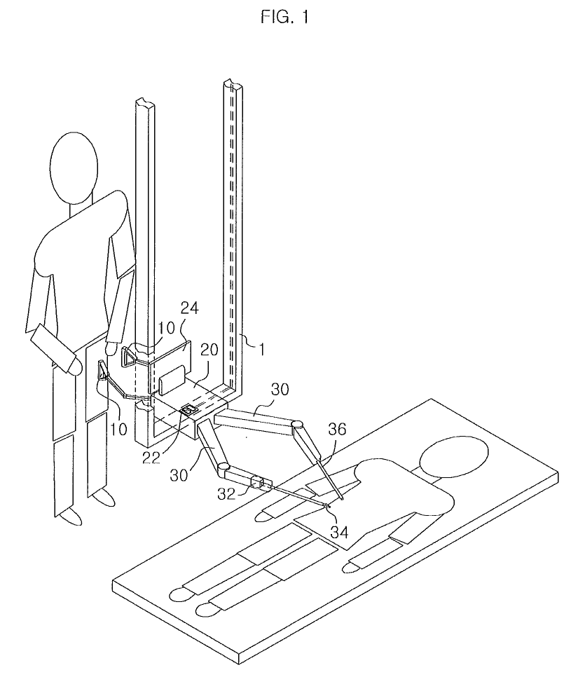

FIG. 1 is a perspective view schematically illustrating a surgical robot according to an embodiment of the present invention. Illustrated in FIG. 1 are a support platform 1, handle units 10, a main body 20, a power supply terminal 22, a monitor 24, robot arms 30, an instrument 32, a manipulation unit 34, and a laparoscope 36.

A feature of this embodiment is that the surgical robot arm is moved and rotated by the operator, while the instrument is manipulated by the robot system, so that the surgical robot can be constructed with a structurally simpler and slimmer form. That is, the distinction between a master robot and a slave robot is removed, and the complicated mechanical composition required to move the arms on the slave robot according to the manipulation on the master robot is omitted, to provide a slimmer structure.

Also, to increase convenience in manipulating the surgical robot based on this embodiment, the surgical robot may be manipulated while supported by a particular support unit, instead of having the operator carry the robot during surgery. Thus, the reliability of the robot surgery can be improved, as the surgical robot may be moved, rotated, and manipulated with greater precision.

FIG. 1 illustrates an example in which the support unit is implemented in the form of a support platform 1. Here, the various power lines and communication lines required by the surgical robot can be connected through the support platform 1, so that the bundles of cables can be organized, and the robot can be conveniently installed and manipulated in the operating room.

The structure of the surgical robot based on this embodiment may include the robot arms 30 on one side of the main body 20 and the handle units 10 for manipulating the robot arms 30 connected to the other side of the main body 20. As described above, the surgical robot based on this embodiment may be an integrated form of the master robot and the slave robot, where the robot arms 30 may be operated by manipulating the handle units 10 connected to the main body 20.

For example, moving a handle unit 10 in one direction can cause a robot arm 30 to move in that one direction accordingly, and rotating the handle unit 10 by a particular angle can cause the robot arm 30 to rotate accordingly, and in this manner, the manipulation on the operator's handles for performing robot surgery may be manifested as is by the movement and rotation of the robot arms 30.

By thus having the robot arms 30 move and rotate in accordance with the movement and rotation of the handle units 10 that are directly connected to the main body 20, the surgical robot can be constructed with a structurally simple form. The manipulation on the handle units 10 for moving and rotating the robot arms 30 will be referred to as a “first manipulation.” The system for moving and rotating the robot arms 30 according to the first manipulation can be implemented using various electronic and/or mechanical systems.

Occasionally, some of the manipulation from among the first manipulation on the handle units 10 can be filtered from being implemented as a movement or rotation on the robot arm 30. If an undesired manipulation is applied to the handle unit 10, for example when the operator's hand is shaking, or when the handle unit 10 inadvertently droops downward because of gravity, etc., a corresponding movement on the robot arm 30 can result in errors in the robot surgery or even accidents.

In case an unintended first manipulation occurs in this way, a filtering unit can be included in the main body 20, to prevent the first manipulation on the handle unit 10 from being translated into a movement on the robot arm 30. The filtering unit can be made as an electrical circuit that detects an unintended first manipulation and prevents a corresponding signal from being transmitted to the robot arm 30, and can also be made as a mechanical composition that prevents a first manipulation from manifesting as a movement or rotation of the robot arm 30 if the first manipulation is smaller than a particular reference value (for instance, when the hands are shaking) or greater than a reference value (for instance, when the handle units 10 droop downwards due to gravity).

A surgical instrument 32 may be mounted on the front end of a robot arm 30. The instrument 32 may be detachably mounted on the robot arm 30 for robot surgery. The instrument 32 may be of various types, according to the type of surgery, and may be a sort of “surgical tool” that may be replaced as necessary during a surgical procedure.

The surgical instrument 32 may generally be composed of a housing that is mounted on the front end of the robot arm 30, a shaft that extends from the housing by a certain length, and a manipulation unit 34 that is coupled to the far end of the shaft. Driving wheels may be coupled to the housing, where the driving wheels may be connected by wires, etc., to the manipulation unit 34, so as to allow the manipulation unit 34 to operate as desired according to the rotations of the driving wheels. For this purpose, actuators may be installed on the front end of the robot arm 30 to rotate the driving wheels coupled to the instrument 32.

In this case, the manipulation unit 34 coupled to the far end of the instrument 32 can be operated by manipulating the handle unit 10 based on this embodiment. The manipulation on the handle unit 10 for operating the manipulation unit 34 will be referred to as a “second manipulation.” Similar to the first manipulation, the second manipulation can also be implemented by a movement or a rotation on the handle, and if necessary, a switch and/or a handle exclusively for inputting a second manipulation can be added to the handle unit 10.

For example, when a switch for a second manipulation is turned off, moving and rotating the handle unit 10 can cause the robot arm 30 to move and rotate accordingly, and when the switch for the second manipulation is turned on, manipulating the handle unit 10 can cause the manipulation unit 34 to perform an action required for surgery.

Furthermore, if there is a handle installed on the handle unit 10 that is dedicated to inputting a second manipulation, the manipulation unit 34 can be made to perform an action required for surgery by manipulating the handle dedicated to second manipulation input. In this case, the handle unit 10 can be used to perform both a first manipulation and a second manipulation simultaneously, and the action of the manipulation unit 34 can be performed at the same time as the movement of the robot arm 30, so that robot surgery can be conducted even in complicated and unusual circumstances.

If a second manipulation is inputted for the handle unit 10, the actuators and the driving wheels coupled to the actuators may each rotate by a particular angle, whereby the manipulation unit 34, which is connected by wires to the driving wheels, may perform an action. However, the mechanism for operating the manipulation unit 34 according to a second manipulation on the handle unit 10 based on the present embodiment is not necessarily limited to that described above, and it is obvious that various electrical/mechanical mechanisms can be applied that are capable of implementing a required action on the manipulation unit 34 for robot surgery.

In the case of the second manipulation also, some of the manipulation can be filtered, so as not to be implemented as an action on the manipulation unit 34, and various types of filtering unit can be included in the main body 20 for this purpose, as in the case of the first manipulation.

As described above, the surgical robot based on this embodiment can be installed on a support platform 1, which corresponds to a sort of structure installed onto the framework of the building, such as on the floor or the ceiling of the operating room. If, for example, the support platform 1 is installed on the ceiling of the operating room, the support platform 1, which may be made from a metal frame, etc., can be secured to the ceiling with anchor bolts, etc. Alternatively, a rail can be secured to the ceiling, and the support platform 1 can be structured as a metal frame that is movable along the rail.

That is, the support platform based on this embodiment does not necessarily have to be secured to the ceiling. The support platform can be installed on the ceiling, but in a movable manner that allows the robot to move within a particular range within the operating room, for example within a range around the operating table on which the patient may lie.

If the support platform is installed on the floor of the operating room, the support platform can be formed as a self-standing frame that has wheels coupled on the bottom. The surgical robot based on this embodiment can be placed on this support platform, so that the robot may readily be moved as necessary to a particular position within the operating room.

If the support platform 1 is secured to the ceiling, the surgical robot installed on the support platform 1 may be supported in a stable manner, allowing greater stability and higher precision during surgery. On the other hand, if the support platform 1 is movable, the surgical robot can also be moved while installed on the support platform 1, and as the surgical robot may be moved as necessary to a suitable position during surgery, the surgery may be performed more smoothly and more speedily.

The main body 20 of the surgical robot based on this embodiment can be secured to the support platform 1 or rotatably and movably installed on the support platform 1. If the main body 20 is movably installed on the support platform 1, the entire surgical robot, i.e. the main body 20 as well as the connected robot arms 30, can be made to move and rotate by manipulating the handle units 10.

The manipulation on the handle units 10 for moving and rotating the entire surgical robot will be referred to as a “third manipulation.” Similar to the first and second manipulations, the third manipulation can also be implemented by a movement or a rotation on the handles, and if necessary, a clutch and/or a switch exclusively for inputting a third manipulation can be added to the handle units 10.

For example, when a clutch for a third manipulation is disengaged, moving and rotating the handle units 10 can correspond to a first manipulation, causing the robot arms 30 to move and rotate accordingly, but when the clutch for the third manipulation is engaged, the first manipulation on the handle units 10 can be mechanically blocked and the handle units 10 can be secured, allowing the user to move and rotate the entire surgical robot by moving and rotating the handle units 10.

Alternatively, if a switch is added for inputting a third manipulation, moving and rotating the handle units 10 when the switch is turned off can cause the robot arms 30 to move and rotate accordingly, but when the switch for the third manipulation is turned on, the signals corresponding to the manipulation of the handle units 10 may not be translated to the movement and rotation of the robot arms 30, allowing the user to move and rotate the surgical robot overall by moving and rotating the handle units 10.

As the entire surgical robot can be moved and rotated in this manner, the surgical robot can be moved when surgery is needed at a position that cannot be reached just by the movement and rotation of the robot arms 30.

In order to generally move and rotate the surgical robot using a third manipulation on the handle units 10, various mechanical components, such as hinges, ball joints, guide rails, etc., can be interposed at the coupling portion between the main body 20 and the support platform 1. A driving motor can be coupled to each of the mechanical components, so that the user may readily move and rotate the surgical robot with only a small amount of force.

That is, the driving motors coupled to the respective components can offset the gravitational force and/or frictional force applied while moving or rotating the robot, so that the user may apply an even amount of force in inputting a third manipulation, regardless of the direction of movement of the surgical robot, whereby the stability and reliability of robot surgery can be improved.

In the case of the third manipulation also, some of the manipulation can be filtered, so as not to be implemented as a movement or rotation of the robot. For example, a stopper, etc., can be installed on the portion where the main body 20 and the support platform 1 are coupled, to allow the surgical robot to readily move or rotate to a predefined position, or to prevent the robot from moving or rotating further.

A power cable or various communication cables can be connected to the surgical robot described above in performing robot surgery. In this case, the surroundings of the robot can be disorganized because of the numerous cables, etc., and the risks of short-circuiting, disconnection, and interference with other medical equipment, etc., may lower the reliability of the robot surgery.

Therefore, the surgical robot based on this embodiment can be implemented such that the power supply or communication lines are connected through the support platform 1. For example, a power supply terminal 22 can be formed on the main body 20, and a corresponding power supply unit can be formed in the support platform 1. Then, during the process of installing the surgical robot on the support platform 1, the surgical robot can be installed in such a way that the power supply terminal 22 is connected to the power supply unit, so that the surgical robot may be supplied with power. This approach can be applied not only to the power supply, but also to the other communication lines, for which various terminals can be formed on the main body 20, and corresponding adapters can be formed in the support platform 1.

A robot surgery procedure may involve the operator inserting a laparoscope 36 in the surgical site and performing surgery while observing the inside of the patient's body through the laparoscope 36. For this purpose, a monitor 24 for outputting image signals can be installed on the surgical robot based on this embodiment. The monitor 24 can be used to display the pulse and heart rate of the patient, the temperature and humidity of the operating room, and the operational status of various equipment, including the surgical robot, as well as the image signals obtained by the laparoscope 36.

Thus, in cases where there are two robot arms 30 connected to the surgical robot based on this embodiment, with a surgical instrument 32 mounted on one of the robot arms 30 and a laparoscope 36 mounted on the other, the operator may perform robot surgery by manipulating the handle unit 10 (i.e. a first manipulation) to insert the instrument 32 and the laparoscope 36 into the body of the patient, and while observing the image obtained by the laparoscope 36 on the monitor 24, manipulating the handle unit 10 (i.e. a second manipulation) to operate the manipulation unit 34.

As described above, the surgical robot based on the present embodiment may have a simpler, slimmer mechanical composition, as the handle units 10 and robot arms 30 are connected directly to the main body 20. Consequently, the robot can be installed in narrower spaces, making it possible to perform robot surgery in situations that did not allow for robot surgery before.

By making the surgical robot portable, the surgical robot can be installed directly beside the patient when conducting surgery, and the reliability of the robot surgery can be improved, as if the operator was performing surgery personally.

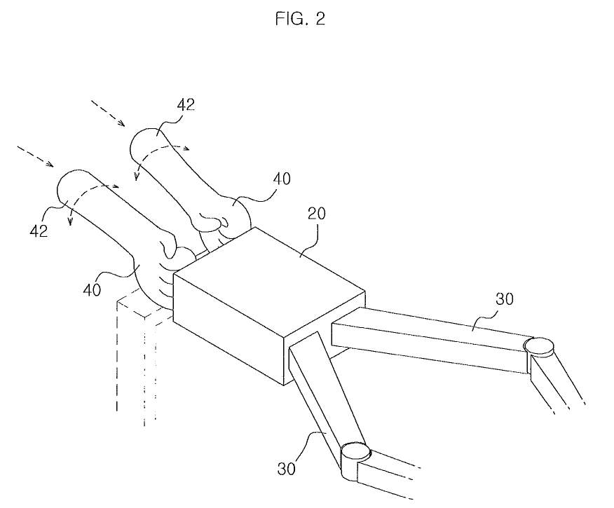

FIG. 2 is a perspective view schematically illustrating a surgical robot according to another embodiment of the present invention. Illustrated in FIG. 2 are a main body 20, robot arms 30, hand guides 40, and arm guides 42.

In this embodiment, the support unit is formed as a set of hand guides 40 that can be fit onto a user's hands. Thus, in addition to forming a support platform 1 as a structure such as those in the previously disclosed embodiment that are secured to the floor or ceiling of the operating room, the handle units 10 can be implemented in a form wearable by the user, to allow more intuitiveness and greater portability in performing robot surgery.

In cases where the support unit is formed as hand guides 40, the handle units 10 can be included in the hand guides 40. The user may fit the hand guides 40 on the user's hands, hold the handle units 10, and then perform a first and/or second manipulation.

Moreover, the hand guides 40 can be implemented in the form of gloves, as illustrated in FIG. 2, with the handle units 10 integrated with the gloves. In this case, various buttons can be formed in portions where the user's fingers would touch, and a rotatable and movable arrangement can be provided in the portions where the user's wrists would touch. The user may fit the gloves based on the present embodiment on the user's hands, just as if the user were wearing gloves, and move the fingers and/or wrists to input a first manipulation or a second manipulation.

The hand guides 40 based on this embodiment can be extended to the elbows or shoulders of the user, in other words, the hand guides 40 can be connected with arm guides 42 that are fit onto the user's arms. Then, the user may be enabled to input a first manipulation or a second manipulation by fitting the hand guides 40 and arm guides 42 onto the user's body and moving the fingers, wrists, elbows, shoulder, etc.

As the support unit based on this embodiment is implemented in the form of hand guides 40, a person using the surgical robot may perform robot surgery in a precise and stable manner, by manipulating the robot as if performing the surgery with the person's own hands.

In cases where the hand guides 40 based on this embodiment are connected to the arm guides 42 to be fitted onto the arms of the user, the second manipulation can be inputted by a movement of the user's finger or wrist, while the first manipulation can be inputted by a movement of the user's arm or shoulder, to provide an intuitive sense that is comparable to performing surgery personally.

The hand guides 40 and arm guides 42 based on this embodiment do not necessarily have to be used in parallel. The surgical robot can just as well include only the hand guides 40 or only the arm guides 42.

For example, if only the arm guides 42 are used, the user may hold the handle units 10, which may be formed as joysticks, etc., and perform a second manipulation, while the user may fit the arm guides 42 on the user's arms to perform a first manipulation. That is, the gloves or arm guides 42, etc., fitted onto the user's arms may be coupled to the main body 20, while the handle units 10 may be implemented in the form of joysticks, etc., or in the form illustrated in FIG. 1.

While this embodiment is described as implementing the support unit in the form of hand guides 40 and arm guides 42, the embodiment is not thus limited, and it is obvious that other forms of support unit can be employed, such as by additionally coupling the support platform 1 of the previously disclosed embodiment to the hand guides 40, etc.

The composition, function, and effects of the surgical robot based on the present embodiment have been described above. The surgical robot based on this embodiment may be implemented in an integrated form, rather than the conventional master/slave structure, and may have the robot arm, instrument, and main body operate in accordance to a first manipulation, second manipulation, and third manipulation on a handle unit.

Also, hand guides and/or arm guides can be installed that include the handle units, so that the first manipulation, second manipulation, and third manipulation may be performed in connection with movements on the user's hands or arms, where the hand guides can be implemented in the form of guide frames, gloves, etc., while the arm guides can be implemented in the form of guide frames, wristlets, etc.

While the present invention has been described with reference to particular embodiments, it is to be appreciated that various changes and modifications can be made by those skilled in the art without departing from the spirit and scope of the present invention as defined by the appended claims.

1. A surgical robot configured to generate, transmit and receive a particular signal in correspondence with a manipulation by a user and perform actions required for surgery comprising:
a handle unit manipulated by a user;
a main body connected to the handle unit and configured to generate a first signal in correspondence with a first manipulation on the handle unit and generate a second signal in correspondence with a second manipulation on the handle unit;
a robot arm connected to the main body and configured to operate by the first signal;
an instrument mounted on a front end of the robot arm; and
a manipulation unit coupled to a far end of the instrument and configured to perform an action required for surgery by the second signal,
wherein the main body is coupled to a support unit.

2. The surgical robot of claim 1, wherein the main body is coupled to the support unit such that the main body is capable of movement and rotation, and the main body is configured to move and rotate in correspondence with a third manipulation on the handle unit.

3. The surgical robot of claim 1, wherein one end of the support unit is installed on a ceiling of an operating room in which the surgical robot is installed.

4. The surgical robot of claim 1, wherein the support unit is formed as a self-standing frame movable within an operating room in which the surgical robot is installed.

5. The surgical robot of claim 1, wherein the main body is equipped with a power supply terminal formed on a portion of the main body coupled to the support unit, and the main body is supplied with electrical power through the support unit.

6. The surgical robot of claim 1, wherein the robot arm is capable of having a laparoscope mounted on a front end thereof, and
the main body has a monitor installed thereon, the monitor configured to output an image signal transmitted from the laparoscope.

7. The surgical robot of claim 1, wherein the support unit is formed as a hand guide, the hand guide including the handle unit and configured to fit onto a user's hand, and
the second manipulation is performed in accordance to a movement of any one or more of a finger and a wrist of the user's hand having the hand guide fitted thereon.

8. The surgical robot of claim 7, wherein the hand guide has an arm guide connected thereto, the arm guide configured to fit onto a user's arm, and
the first manipulation is performed in accordance to a movement of the user's arm.

9. The surgical robot of claim 1, wherein the main body has an arm guide coupled thereto, the arm guide including the handle unit and configured to fit onto a user's arm, and
the first manipulation is performed in accordance to a movement of the user's arm having the arm guide fitted thereon.
